(19) United States
(12) Patent Application Publication
(10) Pub. No.: US 2011/0098791 A1
(43) Pub. Date: Apr. 28, 2011

(54) PROBE FOR LOCAL ANAESTHETIC SYSTEM

(75) Inventor: Dong Woo KIM, Seoul (KR)

(73) Assignees: ELBIO CO., LTD, Seoul (KR); DAEWOONG PHARMACEUTICAL CO., LTD., Seongnam-si (KR); DNCOMPANY, Seoul (KR)

(21) Appl. No.: 11/951,732

(22) Filed: Dec. 6, 2007

(30) Foreign Application Priority Data
Mar. 8, 2007 (KR) 10-2007-0022948

(51) Int. Cl. A61F 7/00

(52) U.S. Cl. 607/104

(57) ABSTRACT

The present invention relates to a probe for a local anaesthetic system, and more particularly, to a probe for a local anaesthetic system which cools a local area of the body to perform a local anaesthetic operation without difficulty. The present invention provides a probe for a local anaesthetic system which cools a local area to be anaesthetized, the probe comprising a cooling pad which has a heat conductive material and a predetermined contacting surface to contact an anaesthetic area; a heat capacity adding means which is liquid to increase a heat capacity of the cooling pad; and a thermoelement which is provided to emit a cool air to at least one of the heat capacity adding means and the cooling pad. The probe performs an anaesthetic operation without pain by instantly cooling a skin layer and blocking a transmission of a pain to the nerve, and performs an anaesthetic operation repeatedly and stably by raising a heat capacity of a cooling pad with a heat capacity adding means.

### CROSS-REFERENCE TO RELATED APPLICATION

This application claims priority from Korean Patent Application No. 10-2007-0022948, filed on Mar. 8, 2007, in the Korean Intellectual Property Office, the disclosure of which is incorporated herein by reference.

### BACKGROUND OF THE INVENTION

1. Field of the Invention

Apparatuses consistent with the present invention relate to a probe for a local anaesthetic system, and more particularly, to a probe for a local anaesthetic system which cools a local area of the body to perform a local anaesthetic operation without difficulty.

2. Description of the Related Art

Generally, if a medical practice such as surgery and treatment or a skin care is expected to give a pain to patients, anaesthetia is performed to patients to block the transmission of the pain to the nerve and remove or lower the pain.

As the anaesthetia is accompanied by side effects, it is used within a limited range such as long-time surgery or treatment, or a dental treatment causing relatively great pain.

Recently, a laser treatment or a simple medial treatment such as removing moles and hair, peeling, Botox injection has been popular. Even though such a simple operation causes considerable pain, there is not a proper anaesthetic method which can be employed easily. Since the simple operation takes relatively short time and focuses on a local part, the anaesthetia is omitted generally. Thus, patients should bear the pain.

Usually, an anaesthetic agent is injected by an injector. In this case, an injector needle adds an extra pain to patients. Further, the injection method requires certain anaesthetic time to achieve anaesthetic effects, which takes an unnecessary standby time, and is inefficient and inconvenient.

An anaesthetic method such as a nitrogen spray has been suggested to solve the foregoing problem of the injection method. However, it is difficult to adjust spray distance, spray time, etc. Also, the nitrogen spray is controversial in that it generates carcinogen, and is barely used. Thus, it is necessary to provide an anaesthetic method which does not cause pain and is used conveniently.

### SUMMARY OF THE INVENTION

Accordingly, it is an aspect of the present invention to provide a probe for a local anaesthetic system which performs an anaesthetic operation with a cooling method causing no pain, particularly cools a local area with an increased heat capacity more stably and efficiently.

An aspect of invention can be achieved by providing a probe for a local anaesthetic system which cools a local area to be anaesthetized, the probe comprising: a cooling pad which is formed of a heat conductive material and has a predetermined contacting surface to contact an anaesthetic area; a heat capacity adding means which is liquid to increase a heat capacity of the cooling pad; and a thermoelement which is provided to emit a cool air to at least one of the heat capacity adding means and the cooling pad.

The cooling pad comprises a metal case which has an accommodation space to accommodate the heat capacity adding means therein.

The probe further comprises a first temperature detector which is provided in the metal case and detects a temperature of the heat capacity adding means to control the thermoelement.

The heat capacity adding means is a liquid which has a high specific heat.

The liquid is a coolant which has an anti freezing solution.

The probe further comprises a plurality of cooling pins which protrudes from an inner surface of the metal case near the thermoelement to increase a cooling efficiency of the heat capacity adding means.

A plurality of metal pieces which has a heat conductive material is inserted into the accommodation space of the metal case to increase a heat capacity.

The probe further comprises a cooling jacket which is provided in a heat-generating part of the thermoelement and accommodates a coolant cooled by a heat exchanging means and circulated by a pump to cool a heat; a second temperature detector which is provided in the heat-generating part of the thermoelement, transmits a detected temperature to a controller to operate the pump and raise the detected temperature to a cooling ending temperature if the detected temperature is higher than a predetermined cooling starting temperature; and a case which accommodates the cooling pad, the heat capacity adding means, the thermoelement and the cooling jacket therein.

The heat capacity adding means is accommodated in an additional container and connected with the cooling pad.

The probe further comprises a third temperature detector which is provided in the contacting surface of the cooling pad and detects a temperature of an anaesthetic area; and an alarming means which generates an alarming signal by comparing a temperature detected by the third temperature detector with a reference temperature.

The alarming signal is in the form of a sound or a light.

Additional aspects and/or advantages of the present invention will be set forth in part in the description which follows and, in part, will be obvious from the description, or may be learned by practice of the present invention.

### BRIEF DESCRIPTION OF THE DRAWINGS

The above and/or other aspects of the present invention will become apparent and more readily appreciated from the following description of the exemplary embodiments, taken in conjunction with the accompany drawings of which:

FIG. 1 is a perspective view of a probe which is installed in a local anaesthetic system according to an exemplary embodiment of the present invention;

FIG. 2 is a block diagram of the probe for the local anaesthetic system according to the exemplary embodiment of the present invention;

FIG. 3 is an exploded perspective view of the probe for the local anaesthetic system according to the exemplary embodiment of the present invention;

FIG. 4A is a partial sectional view of a cooling pad of the probe for the local anaesthetic system according to the exemplary embodiment of the present invention;

FIG. 4B illustrates a cooling pad of the probe for the local anaesthetic system according to a transformative embodiment of the present invention; and

FIG. 4C illustrates a heat capacity adding means of the probe for the local anaesthetic system according to the transformative embodiment of the present invention.

### DETAILED DESCRIPTION OF EXEMPLARY EMBODIMENTS

Hereinafter, exemplary embodiments of the present invention will be described with reference to accompanying drawings, wherein like numerals refer to like elements and repetitive descriptions will be avoided as necessary.

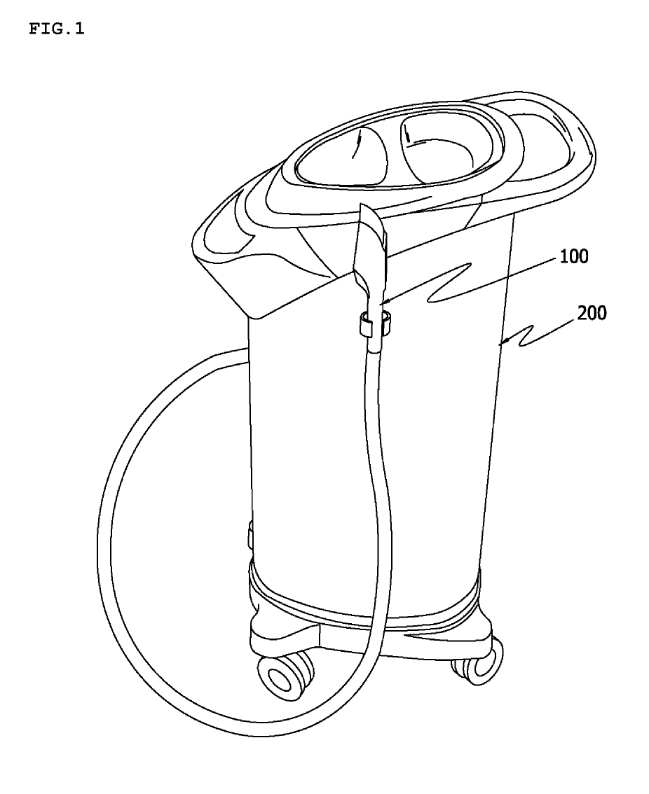

FIG. 1 is a perspective view of a probe which is installed in a local anaesthetic system according to an exemplary embodiment of the present invention.

Referring to FIG. 1, a probe 100 for a local anaesthetic system according to an exemplary embodiment of the present invention contacts a local area of the skin, cools down the skin layer and blocks a transmission of a pain to the nerve to thereby perform an anaesthetic operation. The probe 100 is electrically and physically connected with a main body 200 of the local anaesthetic system through a cable and a coolant line.

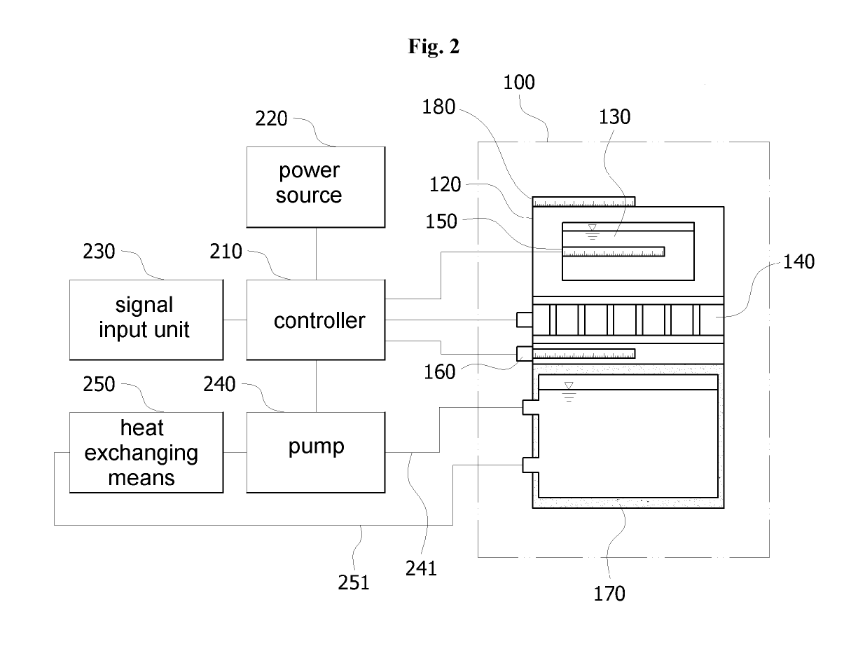

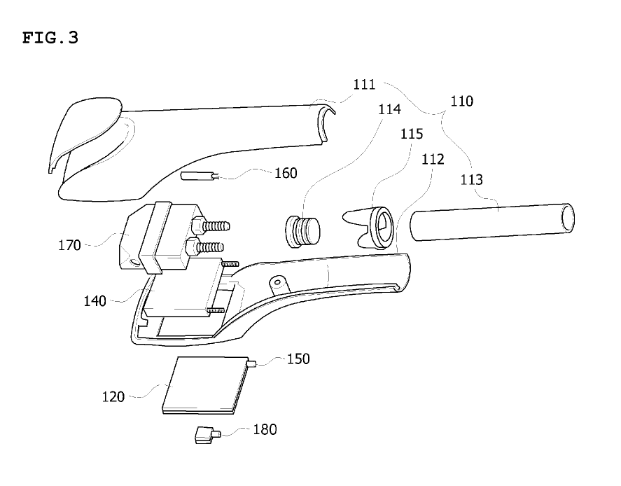

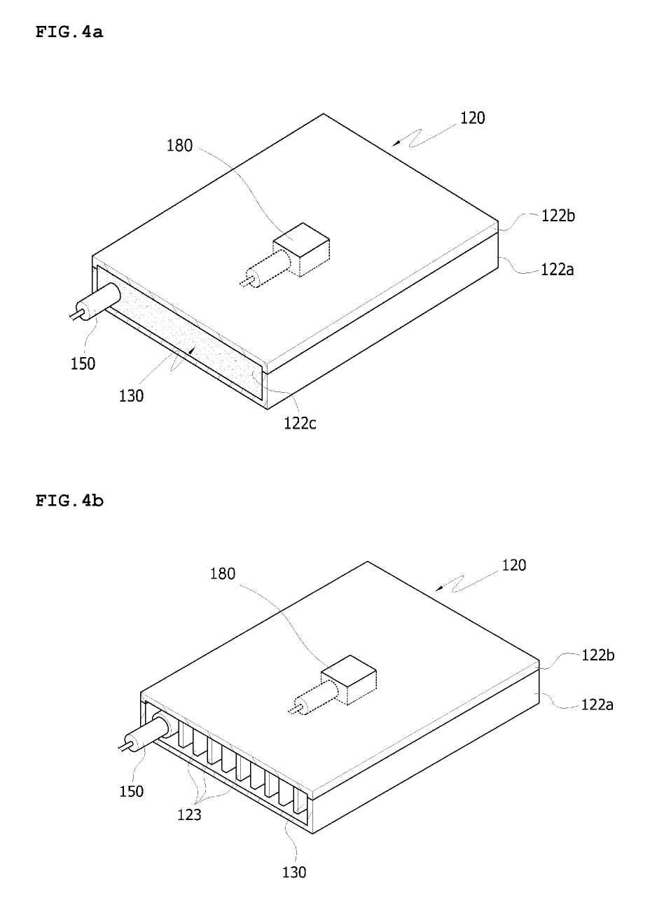

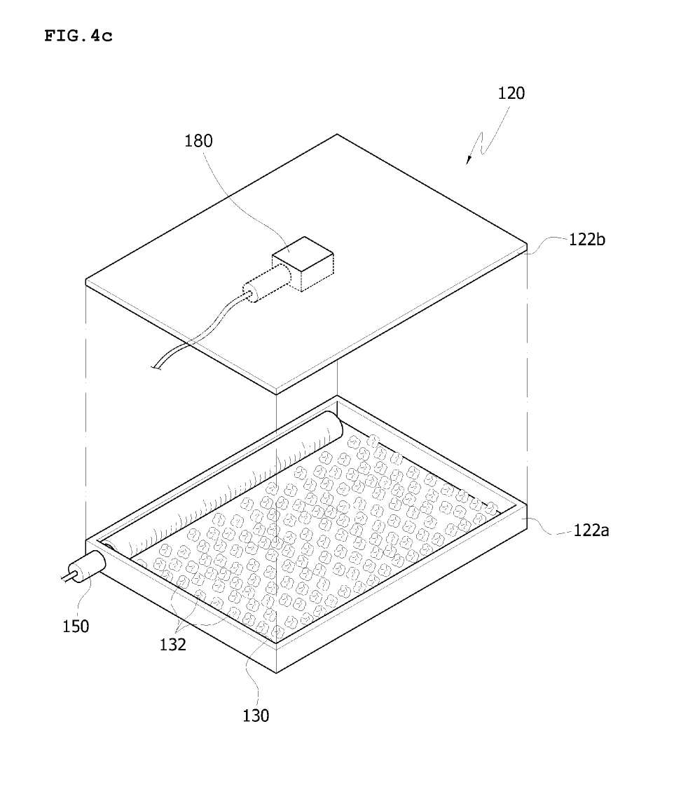

FIG. 2 is a block diagram of the probe 100 for the local anaesthetic system according to the exemplary embodiment of the present invention. FIG. 3 is an exploded perspective view of the probe 100 for the local anaesthetic system according to the exemplary embodiment of the present invention. FIG. 4A is a partial sectional view of a cooling pad 120 of the probe 100 for the local anaesthetic system according to the exemplary embodiment of the present invention.

Referring to FIGS. 2 and 3, the probe 100 includes a grip to be grabbed by a user. A user may grab the grip and apply the probe 100 to an anaesthetic area of the body. A case 110 includes the cooling pad 120, a heat capacity adding means 130, a thermoelement 140, a first temperature detector 150, a second temperature detector 160 and a cooling jacket 170.

The cooling pad 120 includes a highly-heat-conductive material to efficiently transmit a cool air generated by the thermoelement 140. The cooling pad 120 has a predetermined width to contact a local area of the body, and includes a contacting surface to contact the body stably.

As shown in FIG. 4A, the cooling pad 120 includes a metal case 122 which has an accommodation space to accommodate the heat capacity adding means 130 therein. The metal case 122 includes an aluminum material which has relatively good heat conductivity. The metal case 122 has substantially a hexahedron shape, and preferably a pad having a relatively thin thickness.

The cooling pad 120 may include a pad main body 122a which has an accommodation groove to accommodate the heat capacity adding means 130 therein, and a pad cover 122b which is tightly coupled with the accommodation groove 122c of the pad main body 122a accommodating the heat capacity adding means 130, but not limited thereto. Alternatively, an injection hole of the pad main body 122a may be airtight after the heat capacity adding means 130 is injected through the injection hole.

The material of the cooling pad 120 is not limited to aluminum. Alternatively, the cooling pad 120 may vary as long as it is highly heat conductive, light and freezing-resistant.

The heat capacity adding means 130 is provided to add a heat capacity to the cooling pad 120. The term “to add a heat capacity” means that a temperature of the cooling pad 120 is prevented from drastically rising and the cooling pad 120 is not easily warmed up when contacting the skin. The conventional cooling pad is warmed up in a short time when contacting the skin and absorbing body heat. In this case, the anaesthetic effect is lowered and the cooling pad may not be used repetitively.

The thickness of the cooling pad 120 may be enlarged to raise the heat capacity. However, such a method increases the thickness and less efficient. Also, the overall size of the probe 100 increases according to the increased volume of the cooling pad 120. Then, it takes more time to cool the cooling pad 120, and consecutive anaesthesia is impossible.

Thus, the heat capacity adding means 130 according to the present embodiment may include various liquid having high specific heat, and preferably a coolant. The coolant has relatively high specific heat within a determined mass, has high stability and does not cost much. The heat capacity is proportional to specific heat and mass.

The heat capacity adding means 130 preferably includes an anti freezing solution not to damage the cooling pad 120 due to expansion of a frozen coolant during a cooling process of the thermoelement 140. The anti freezing solution may include calcium chloride, magnesium chloride, ethylene glycol, ethyl alcohol, etc.

The heat capacity adding means 130 may be accommodated in an additional container having heat conductivity and freezing resistance to be disposed between the cooling pad 120 and the thermoelement 140. More preferably, the heat capacity adding means 130 is accommodated in the cooling pad 120 having the accommodation space therein, in consideration of the cooling efficiency, a size of the probe 100 and simplification of the configuration.

The first temperature detector 150 is disposed in the metal case 122 to detect a temperature of the heat capacity adding means 130. The detected temperature is transmitted to a controller 210. If the temperature which is detected by the first temperature detector 150 and transmitted to the controller 210 is higher than a predetermined threshold value, the thermoelement 140 is driven to cool the heat capacity adding means 130 and raise the detected temperature to a target temperature.

A cooling part of the thermoelement 140 is disposed toward the cooling pad 120 to emit a cool air to the heat capacity adding means 130 and the cooling pad 120. Typically, the thermoelement 140 includes a Peltier element in which a first side emits a cool air and a second side emits a heat by supplying power. The capacity and standard of the thermoelement 140 may vary in a heat capacity of the cooling pad 120 that is determined according to usage purposes of the probe 100.

The second temperature detector 160 is provided in a heat-generating part of the thermoelement 140. A temperature detecting signal of the heat generating part is transmitted to the controller 210. If the temperature transmitted to the controller 210 is higher than a predetermined cooling starting temperature, a pump 240 is driven to circulate the coolant cooled by a heat exchanging means 250 until the detected temperature rises to a cooling ending temperature.

If the pump 240 continuously operates to circulate the coolant, the second temperature detector 150 may be omitted.

The cooling jacket 170 is disposed in the heat-generating part of the thermoelement 140 and accommodates the coolant which is cooled by the heat exchanging means 250 and circulated by the pump 240 to cool the emitted heat. The cooling jacket 170 has an accommodation space to accommodate the coolant therein. The cooling jacket 170 has a substantially hexahedron shape, and includes a pair of nozzles to be connected with a coolant supplying line 251 and a coolant discharging line 241.

The case 110 includes upper and lower cases 111 and 112 which are bent downwardly from the grip and form an accommodation space, a connection pipe 113 which is connected with a grip connector of the upper and lower cases 111 and 112 and has the coolant supplying line 251 and the coolant discharging line 241 therein, and front and rear connectors 114 and 115 which connect the connection pipe 113 to the upper and lower cases 111 and 112.

The cooling pad 120, the thermoelement 140, the second temperature detector 160 and the cooling jacket 170 are provided in the lower bent part of the upper and lower cases 111 and 112. The shape and size of the case 110 is not limited to that described above. Alternatively, the shape and size of the case 110 may vary in elements changed by usage purposes and cooling capacity and in other design factors.

As shown in FIGS. 1 and 2, the main body 200 which is electrically and physically connected with the probe 100 for the local anaesthetic system according to the present embodiment includes the pump 240 which is connected with the coolant discharging line 241 connected with one of the nozzles of the cooling jacket 170, and the heat exchanging means 250 which has a radiator to be connected with the pump 240, perform heat exchange of the supplied coolant and supply the coolant to the cooling jacket 170 through the coolant supplying line 251. The main body 200 further includes the controller 210 electrically connected with the thermoelement 140, the first temperature detector 150 and the second temperature detector 160, and a power source 220 and a signal input unit 230.

FIG. 4B illustrates a cooling pad 120 of a probe 100 for a local anaesthetic system according to a transformative embodiment of the present invention. As shown therein, a metal case 122 forms an accommodation space to accommodate a heat capacity adding means 130 such as a coolant. A cooling air which is supplied by a thermoelement 140 is more efficiently emitted to the heat capacity adding means 130. Thus, a heat capacity of the cooling pad 120 increases while cooling time is reduced.

That is, a plurality of cooling pins 123 protrudes from the inner surface of the metal case 122 near the thermoelement 140 to increase a contacting area with the accommodated heat capacity adding means 130. Thus, the cooling time of the heat capacity adding means 130 is reduced and cooling efficiency is enhanced.

The cooling pins 123 have a large surface area generating a cool air. The cooling pins 123 are plurally provided within a certain range.

FIG. 4C illustrates the heat capacity adding means 130 of the probe 100 for the local anaesthetic system according to a transformative embodiment of the present invention. As shown therein, a plurality of metal pieces 132 is provided in the cooling pad 120 other than the coolant as a heat capacity adding means 130. The metal pieces 132 are inserted into the metal case 122 to contact the coolant and increase the heat capacity further.

The metal pieces 132 may be formed like metal beads, and preferably include metal pieces having a large surface area to receive the cool air from the coolant and reduce the cooling time.

The ultimate purpose of the present invention is to lower the temperature of the local area of the body to a certain temperature and below and block a transmission of a pain to the nerve to achieve anaesthetic effects. In this regard, a temperature adjustment in the anaesthetic area is most critical.

The excessive cooling may make patients unpleasant, and may cause a pain. Conversely, the deficient cooling may lead to inefficient anaesthetia and give a pain to patients during an operation. The actual temperature of the anaesthetic area is particularly important. The temperature of the anaesthetic area, instead of the temperature of the cooling pad 120, should be estimated to be precisely controlled.

As shown in FIG. 4, a third temperature detector 180 is provided in the contacting surface of the cooling pad 120. The third temperature detector 180 is provided in the cooling pad 120 so that a sensing part of the third temperature detector 180 is in the same level with the contacting surface of the cooling pad 120.

A user may recognize whether the anaesthetic operation is completed, with a temperature detected by the third temperature detector 180.

That is, if the temperature of the anaesthetic area is lowered to a reference temperature and below, an alarming signal may be generated to inform a user of the completion of the anaesthetia or a necessity of the cooling suspension. The alarming signal may vary including a flickering light and a chime bell.

The reference temperature at which the alarming signal is generated may depend on ages, genders, etc. The reference temperature may be properly determined by various experiments and database buildup.

Hereinafter, an operation of the probe 100 for the local anaesthetic system according to the exemplary embodiment of the present invention will be described.

As shown in FIGS. 1 and 3, the heat capacity adding means 130 and the first temperature detector 150 are provided in the lower part of the case 110. The cooling pad 120 including the third temperature detector 180, the thermoelement 130, the second temperature detector 160 and the cooling jacket 140 are sequentially provided in the contacting surface of the case 110, thereby completing the probe 100.

The coolant supplying line 251 and the coolant discharging line 241 connected with the nozzles of the cooling jacket 140 of the probe 100 are connected with the pump 240 and the heat exchanging means 250 of the main body 200. The thermoelement 140, the first temperature detector 150 and the second temperature detector 160 are electrically connected with the power source 220 and the controller 210 of the main body 200 through a cable, thereby completing the local anaesthetic system.

Hereinafter, a process of performing the anaesthetic operation to the local area of the body with the local anaesthetic system will be described. First, if the thermoelement 140 operates by manipulating the signal input unit 230, the cool air is emitted to the cooling pad 120 to cool the cooling pad 120. At the same time, the heat capacity adding means 130 which is accommodated in the accommodation space of the cooling pad 120 is cooled.

The heat capacity to cool the skin includes the heat capacity of the heat capacity adding means 130 of the cooling pad 120, as well as the heat capacity of the cooling pad 120, thereby providing sufficient cooling effect.

As shown in FIG. 4B, if the plurality of cooling pins 123 is formed in the thermoelement 140 of the metal case 122, the cool air is efficiently transmitted from the thermoelement 140 to the accommodation space of the heat capacity adding means 130, and increases the contacting surface area with the heat capacity adding means 130 to thereby raise the heat capacity and reduce the cooling time. As shown in FIG. 4C, if the plurality of metal pieces 130 is inserted into the metal case 122, the heat capacity may be further raised.

If the cooling pad 120 is cooled, the probe 100 is moved to the local anaesthetic area to contact the skin. Then, the skin layer is anaesthetized through the cooling operation. If the cooling operation is repeatedly performed, the temperature of the cooling pad 120 rises as the cooling pad 120 absorbs heat from the skin. Also, the temperature of the heat capacity adding means 130 in the cooling pad 120 rises.

The temperature of the heat capacity adding means 130 is detected by the first temperature detector 150 to be transmitted to the controller 210. If the detected temperature is higher than the predetermined threshold value, the thermoelement 140 is driven to cool the heat capacity adding means 130 and raise the detected temperature to the target temperature. Thus, the heat capacity for the cooling operation is consistent.

If the thermoelement 140 is driven to cool the cooling pad 120, the heat is emitted to the heat-generating part. The heat is detected by the second temperature detector 160 and transmitted to the controller 210. If the detected temperature is higher than the predetermined cooling starting temperature, the pump 240 is driven to circulate the coolant cooled by the heat exchanging means 250 within the cooling jacket 170 and raise the detected temperature to the cooling ending temperature. Then, the thermoelement 140 is prevented from being overheated and the cooling operation is performed continuously and stably.

described above, the present invention provides a probe for a local anaesthetic system which performs an anaesthetic operation without pain by instantly cooling a skin layer and blocking a transmission of a pain to the nerve, and performs an anaesthetic operation repeatedly and stably by raising a heat capacity of a cooling pad with a heat capacity adding means.

Although a few exemplary embodiments of the present invention have been shown and described, it will be appreciated by those skilled in the art that changes may be made in these exemplary embodiments without departing from the principles and spirit of the invention, the scope of which is defined in the appended claims and their equivalents.

1. A probe for a local anaesthetic system which cools a local area to be anaesthetized, the probe comprising:
a cooling pad which is formed of a heat conductive material and has a predetermined contacting surface to contact an anaesthetic area;
a heat capacity adding means which is liquid to increase a heat capacity of the cooling pad; and
a thermoelement which is provided to emit a cool air to at least one of the heat capacity adding means and the cooling pad.

2. The probe according to claim 1, wherein the cooling pad comprises a metal case which has an accommodation space to accommodate the heat capacity adding means therein.

3. The probe according to claim 2, further comprising a first temperature detector which is provided in the metal case and detects a temperature of the heat capacity adding means to control the thermoelement.

4. The probe according to claim 1, wherein the heat capacity adding means is a liquid which has a high specific heat.

5. The probe according to claim 4, wherein the liquid is a coolant which has an anti freezing solution.

6. The probe according to the claim 4, further comprising a plurality of cooling pins which protrudes from an inner surface of the metal case near the thermoelement to increase a cooling efficiency of the heat capacity adding means.

7. The probe according to claim 4, wherein a plurality of metal pieces which has a heat conductive material is inserted into the accommodation space of the metal case to increase a heat capacity.

8. The probe according to claim 4, further comprising a cooling jacket which is provided in a heat-generating part of the thermoelement and accommodates a coolant cooled by a heat exchanging means and circulated by a pump to cool a heat;
a second temperature detector which is provided in the heat-generating part of the thermoelement, transmits a detected temperature to a controller to operate the pump and raise the detected temperature to a cooling ending temperature if the detected temperature is higher than a predetermined cooling starting temperature; and
a case which accommodates the cooling pad, the heat capacity adding means, the thermoelement and the cooling jacket therein.

9. The probe according to claim 1, wherein the heat capacity adding means is accommodated in an additional container and connected with the cooling pad.

10. The probe according to claim 4, further comprising a third temperature detector which is provided in the contacting surface of the cooling pad and detects a temperature of an anaesthetic area; and
an alarming means which generates an alarming signal by comparing a temperature detected by the third temperature detector with a reference temperature.

11. The probe according to claim 10, wherein the alarming signal is in the form of a sound or a light.

12. The probe according to claim 2, wherein the heat capacity adding means is a liquid which has a high specific heat.

13. The probe according to claim 3, wherein the heat capacity adding means is a liquid which has a high specific heat.
